(19) United States
(12) Patent Application Publication
(10) Pub. No.: US 2013/0344121 A1
(43) Pub. Date: Dec. 26, 2013

(54) COSMETIC COMPOSITION FOR MAKE-UP CONTAINING SILICONE ELASTOMER

(71) Applicants: Sung Yong Kim, Anyang-si (KR); Jeong Ho Bae, Hwaseong-si (KR); Myeong Sam Park, Seoul (KR)

(72) Inventors: Sung Yong Kim, Anyang-si (KR); Jeong Ho Bae, Hwaseong-si (KR); Myeong Sam Park, Seoul (KR)

(73) Assignee: COSMAX CO., LTD., Hwaseong-si (KR)

(21) Appl. No.: 14/003,889

(22) PCT Filed: Mar. 9, 2012
(86) PCT No.: PCT/KR2012/001738

§ 371 Date: Sep. 9, 2013

(30) Foreign Application Priority Data
Mar. 11, 2011 (KR) 10-2011-0021639

(51) Int. Cl. A61K 8/891; A61Q 1/10; A61Q 1/08; A61Q 1/00

(52) U.S. Cl. CPC A61K 8/891; A61Q 1/00; A61Q 1/10; A61Q 1/08
USPC 424/401; 424/78.02

(57) ABSTRACT

Disclosed is an elastic make-up cosmetic composition containing a silicone elastomer. More particularly, the make-up cosmetic composition includes a powder type silicone elastomer, powder extender pigment, hydrocarbon gel, anhydrous silicone oil and organic dispersant oil. A silicone elastomer of a surface-treated powder type and a polymer are used to improve the dispersibility of silicone elastomer when producing a cosmetic composition. As a result, it is possible to provide a make-up cosmetic composition having a silky and soft elastic feel. The resultant cosmetic composition is adhered to the skin smoothly, has a good elastic feel and spreadability and shows little dust flying.

### TECHNICAL FIELD

The present invention relates to an elastic make-up cosmetic composition containing a silicone elastomer. More particularly, the present invention relates to a make-up cosmetic composition, including a powder type silicone elastomer, powder extender pigment, hydrocarbon gel, anhydrous silicone oil and organic dispersant oil.

### BACKGROUND ART

In general, compression molding used widely in powder make-up products is a conventional technology wherein an extender pigment, such as talc, mica, sericite, silica, calcium carbonate, kaolin or starch, is used in combination with an inorganic dye, such as metal iron oxides, navy blue or Purssian blue, an organic dye, such as a tar dye, and a pearlescent pigment utilizing the refractive index of a pigment to impart a color tone, and an oil is used as a binder to control the amount applied to the skin and to improve the compression moldability. In the case of color make-up, the type and amount of oil (ester-based oil and silicone oil) used therefor are varied or the particle size of powder is controlled to adjust the skin covering effect, depending on the type of make-up ranging from transparent covering make-up that allows one to see the skin texture to thick covering make-up that allows complete covering of skin defects. In the case of a product requiring expression of a strong color tone, such as an eye shadow, oil and powder having a high covering effect are used widely. However, powder type face powder and cheek blusher have a relatively low amount of inorganic and organic dyes in order to allow the skin to be seen natural. For transparent skin make-up, the amount of a high-refractive index material, such as titanium dioxide or zinc oxide, or diluent powder, such as silica or nylon-12 powder is reduced or plate-like mica having a high aspect ratio (width/thickness ratio) or a material, such as a silicone elastomer, having high oil absorptivity to absorb sebum and sweat is used together during formulation. In addition, an oil binder including silicone oil formed by dispersing a silicone elastomer into silicone is used to provide a make-up powder cosmetic agent having high transparency and consistency. Silicone oils may be classified into volatile oils and non-volatile oils. In the case of compressed powder cosmetic agents, non-volatile oils are used widely. Non-volatile oils are advantageous in that they are less skin irritating, form a thin film after make-up and have low affinity to water or ester-based oils so that they are immiscible with ester oils that are main ingredients of sweat or sebum secretion, and thus improve make-up consistency and impart a soft feel of use typical to silicone upon application. When compressed powder products include volatile silicone, they may undergo deformation of their contents as the volatile components evaporate with time. Such compressed powder products containing a silicone elastomer are advantageous in that they absorb various types of oils, correct the skin condition and form a thin coating film. However, they have viscoelasticity, cannot be fused with talc, mica, silica, metal stone soap, calcium carbonate, etc., used as extender pigments, and are brittle even under small impact, so that they have molding instability.

Therefore, the present inventors have conducted many studies to overcome the above-mentioned problems according to the related art. As a result, we have found that when a solvent in which a silicone oil and polymer are dispersed together with an olefin oil and ester-based oil is mixed with surface-treated silicone elastomer powder, it is possible to improve reagglomeration, aggregation and moldability of silicone elastomer while not adversely affecting the elastic feel, thereby providing an elastic cosmetic composition having a high transparency and blooming effect.

### DISCLOSURE

### Technical Problem

A technical problem to be solved by the present invention is to provide a make-up cosmetic composition having a silky and soft elastic feel.

### Technical Solution

In one general aspect, there is provided a make-up cosmetic composition, including 2-9 wt % of a powder type silicone elastomer, 40-50 wt % of a powder extender pigment, 7-12 wt % of a hydrocarbon gel, 7-14 wt % of anhydrous silicone oil and 20-25 wt % of organic dispersant oil.

Preferably, the powder type silicone elastomer may be a silicone elastomer surface-treated with silica, lauroyl lysine, alumina or titanium dioxide. Such a silicone elastomer may be provided not as a gel type causing agglomeration but as a powder type by being coated with 1.0-5.0 wt % of the above-mentioned materials.

The powder type silicone elastomer used herein may be commercially available one, and particular examples thereof include but are not limited to: EP-9261 TI powder (Dimethicone/Vinyl Dimethicone Crosspolymer treated with titanium dioxide), EP-9289 LL powder (Dimethicone/Vinyl Dimethicone Crosspolymer treated with lauroyl lysine), EP-9293 Al powder (Dimethicone/Vinyl Dimethicone Crosspolymer treated with alumina), and DC 9701 powder (Dimethicone/Vinyl Dimethicone Crosspolymer treated with silica), all of which are available from Dow Corning.

The silicone elastomer is white spherical powder and has viscoelasticity and a silky and cushiony feel. However, the silicone elastomer undergoes severe self-agglomeration and aggregation with the lapse of time, and is highly brittle under impact when provided in the form of compressed powder. Thus, according to the present invention, a solvent formed by dispersing a silicone oil (dimethicone crosspolymer dispersed in silicone) and a high-molecular weight copolymer (hydrocarbon gel) into an olefin oil and ester-based oil is mixed with silicone elastomer powder to improve reagglomeration, aggregation and moldability of the silicone elastomer, while not adversely affecting an elastic feel.

When formulating the cosmetic composition according to the present invention, the powder type silicone elastomer may be used preferably in an amount of 2-9 wt %. When the amount is less than 2 wt %, it is not possible to obtain a sufficient elastic feel. On the other hand, when the amount is larger than 9 wt %, a feel of use and moldability may be degraded.

In the cosmetic composition according to the present invention, the powder extender pigment includes any powder type pigment added to cosmetic agents in order to impart a color, touch feel or texture thereto, and serves to improve the stability of a powder product and functions as a diluent. In general, the powder extender pigment includes talc, mica, silica, titanium dioxide, zinc oxide and boron nitride. However, as used herein, the powder extender pigment also includes a pearlescent pigment and dye that are powder type coloring pigments.

In the cosmetic composition according to the present invention, the powder extender pigment includes at least one selected from the group consisting of talc, mica, silica, titanium dioxide, zinc oxide, boron nitride, pearlescent pigments and dyes.

In the cosmetic composition according to the present invention, a combination of silica with powder type silicone elastomer is used to assist the dispersion of powder, to absorb sebum by virtue of high oil absorptivity and to impart an elastic feel. In addition, a pearlescent pigment is incorporated to impart a soft elastic feel. The pearlescent pigment includes mica coated with titanium dioxide (mica & titanium dioxide), wherein mica is present in the form of thin and transparent plates to provide an elastic feel. Therefore, such a pearlescent pigment enhances the elastic feel of a formulation, thereby contributing to stabilization of the formulation. The pearlescent pigment shows a difference in gloss depending on its particle size, and may be used preferably in an amount of 6-8 wt %. For a desired formulation, non-coated mica and alkylsilane-coated mica may be used in an amount of 9-15 wt % and 4-6 wt %, respectively.

In the cosmetic composition according to the present invention, the hydrocarbon gel is obtained by dispersing an ethylene/propylene/styrene copolymer or butylene/ethylene/styrene copolymer into a mineral oil, isoparaffin or isopropyl palmitate solvent. The hydrocarbon gel is a high-molecular weight copolymer, and thus imparts an elastic feel to the cosmetic composition.

The hydrocarbon gel has a viscosity of 50,000-70,000 cPs. Particular examples of the hydrocarbon gel include but are not limited to VERSAGEL M500 or ME500, MP 750 series available from Penreco Co. In addition, polybutene or polyisobutene may be added to improve the molding stability and adhesion to the skin and to impart gloss.

The hydrocarbon gel used herein has high viscosity and cannot be used directly as a binder for powder. Also, it is not dispersed well in anhydrous oil. Thus, the hydrocarbon gel is used by dispersing it into olefin oil in combination with ester-based oil. Herein, anhydrous silicone oil is used to impart a wet and silky feel and to avoid a complicated vacuum packaging step in molding and product packaging processes by virtue of its presence as slurry-like powder. Further, in the composition according to the present invention, 7-12 wt % of a hydrocarbon gel is dispersed into organic dispersant oil in combination with 7-14 wt % of anhydrous silicone oil. Use of the hydrocarbon gel enhances an elastic feel.

In the composition according to the present invention, the anhydrous silicone oil is Dimethicone/Vinyl dimethicone Crosspolymer dispersed in dimethicone.

The anhydrous silicone oil may be commercially available one, and particular examples thereof include but are not limited to DC 9401 silicone elastomer blend available from Dow Corning and KSG-16 available from Shinetsu, Japan.

In the composition according to the present invention, the organic dispersant oil is ester-based oil and/or olefin oil.

The cosmetic composition according to the present invention may be obtained by mixing its ingredients together to form elastic flour slurry and molding the slurry into a desired formulation by using an extruder or molding machine.

The cosmetic composition according to the related art is provided as a compressed powder or oily cream formulation. A powder formulation has a hard feel of use and shows dust flying, thereby providing poor adhesion. A cream formulation has a wet feel of use and shows no dust flying, but is disadvantageous in that a constant temperature should be maintained during molding and many different forms, such as a dome-like form, cannot be molded.

However, according to the cosmetic composition of the present invention, powdery raw material 1 is mixed with oily raw material 2 at a ratio of about 50-60:40-50 to provide elastic flour slurry, and then the slurry is drawn through an extruder and is put on an aluminum pan to be molded by a mold having a desired shape. In this manner, it is possible to provide many different forms ranging from a planar shape to a dome-like shape.

According to an embodiment, the cosmetic composition is a blusher or eye shadow.

### Advantageous Effects

According to the embodiments of the present invention, a silicone elastomer of a surface-treated powder type and a high-molecular weight copolymer are used in combination to improve the dispersibility of silicone elastomer when producing a cosmetic composition. As a result, it is possible to provide a make-up cosmetic composition having a silky and soft elastic feel. The resultant cosmetic composition is adhered to the skin smoothly, has a good elastic feel and spreadability and shows little dust flying.

### BEST MODE

Exemplary embodiments now will be described more fully hereinafter with reference to the accompanying drawings, in which exemplary embodiments are shown. This disclosure may, however, be embodied in many different forms and should not be construed as limited to the exemplary embodiments set forth therein.

### Examples 1-4

### Preparation of Cosmetic Composition (Blusher)

Powdery raw material 1 and oily raw material 2 as shown in the following Table 1 are used to provide a blusher. The powder mixture of raw material 1 includes talc, mica, silica, coated silica, a spherical silicone elastomer (Dimethicone/Vinyl dimethicone Crosspolymer coated or non-coated with silica), pearlescent pigment (mica & titanium dioxide), boron nitride powder, titanium dioxide, and a dye. In addition, raw material 2, an oil binder of the powder, includes silicone oil (Dimethicone/Vinyl dimethicone Crosspolymer dispersed Dimethicone), a hydrocarbon gel (ethylene/propylene/styrene copolymer or butylene/ethylene/styrene copolymer), olefin oil (hydrogenated C6-14 olefin polymer), and ester-based oil (hexyl laurate and diisostearyl maleate).

TABLE 1
Blusher Ingredients and Amounts thereof

| Ingredients | | Form |
|---|---|---|
| Raw material 1 | Powder type silicone elastomer, | Powder |
| | powder extender pigment | |
| Raw material 2 | Silicone oil, hydrocarbon gel, | Oil |
| | organic dispersant oil | |

### Comparative Examples 1-3

### Preparation of Cosmetic Composition (Blusher)

The same materials as Examples 1-4 are used to provide a blusher, except that the content of silicone elastomer used in raw material 1 and coating type of silicone elastomer are changed.

The amounts of ingredients forming the blushers according to Examples and Comparative Examples are shown in the following Table 2 in more detail.

TABLE 2
Blusher Compositions

| | | Comp. | | | Comp. | | | Comp. |
|---|---|---|---|---|---|---|---|---|
| | | Ex. 1 | Ex. 1 | Ex. 2 | Ex. 2 | Ex. 3 | Ex. 4 | Ex. 3 |
| Raw | Talc | To 100 | To 100 | To 100 | To 100 | To 100 | To 100 | To 100 |
| material 1 | Mica | 10.0 | 10.0 | 10.0 | 10.0 | 10.0 | 10.0 | 10.0 |
| | Silica | 2.0 | 2.0 | 2.0 | 2.0 | 2.0 | 2.0 | 2.0 |
| | Silicone elastomer | 1.0 | 3.0 | 5.0 | 10.0 | — | — | — |
| | Silicone elastomer- | — | — | — | — | 3.0 | 7.0 | 10.0 |
| | coated | | | | | | | |
| | Coated mica | 5.0 | 5.0 | 5.0 | 5.0 | 5.0 | 5.0 | 5.0 |
| | Preservative | 1.0 | 1.0 | 1.0 | 1.0 | 1.0 | 1.0 | 1.0 |
| | Magnesium stearate | 1.0 | 1.0 | 1.0 | 1.0 | 1.0 | 1.0 | 1.0 |
| | Colorant | 1.0 | 1.0 | 1.0 | 1.0 | 1.0 | 1.0 | 1.0 |
| | Mica & Titanium dioxide | 7.0 | 7.0 | 7.0 | 7.0 | 7.0 | 7.0 | 7.0 |
| | Boron nitride | 1.0 | 1.0 | 1.0 | 1.0 | 1.0 | 1.0 | 1.0 |
| Raw | Ethylene/propylene/styrene | 7.5 | 7.5 | 7.5 | 7.5 | 7.5 | 7.5 | 7.5 |
| material 2 | copolymer | | | | | | | |
| | Butylene/ethylene/styrene | | | | | | | |
| | copolymer | | | | | | | |
| | Hexyl laurate | 5.0 | 5.0 | 5.0 | 5.0 | 5.0 | 5.0 | 5.0 |
| | Hydrogenated C6-14 | 9.0 | 9.0 | 9.0 | 9.0 | 9.0 | 9.0 | 9.0 |
| | olefin polymer | | | | | | | |
| | Diisostaryl maleate | 9.0 | 9.0 | 9.0 | 9.0 | 9.0 | 9.0 | 9.0 |
| | Silicone oil | 10.0 | 10.0 | 10.0 | 10.0 | 10.0 | 10.0 | 10.0 |

The blusher is provided by using the above-described ingredients as follows.

According to the compositional ratio as described above, 50-60 wt % of raw material 1 (powder part) is introduced into a Henschel mixer and mixed at a low speed (400-700 rpm) and high speed (700-1500 rpm). Then, the mixture is introduced into a ribbon mixer or multi-functional mixer, and 40-50 wt % of raw material 2 (oil part) is sprayed thereto uniformly while they are mixing at 400 rpm, followed by kneading. After determining whether or not the oil binder including the silicone oil and high-molecular weight copolymer is dispersed sufficiently, the resultant mixture is subjected to molding under a pressure of 5-10 kgf/cm² for 1.0-2.0 seconds.

### Examples 5-7 and Comparative Example 4-7

### Preparation of Cosmetic Composition (Blusher)

The same materials as Examples 1-4 are used to provide a blusher, except that the content of silicone gel used in raw material 2 is changed.

The amounts of ingredients forming the blushers according to Examples and Comparative Examples are shown in the following Table 3 in more detail. The blushers are obtained by using the ingredients as described below in the same manner as described above.

TABLE 3
Blusher Compositions

| | | Comp. | Comp. | | | | Comp. | Comp. |
|---|---|---|---|---|---|---|---|---|
| | | Ex. 4 | Ex. 5 | Ex. 5 | Ex. 6 | Ex. 7 | Ex. 6 | Ex. 7 |
| Raw | Talc | To 100 | To 100 | To 100 | To 100 | To 100 | To 100 | To 100 |
| material 1 | Mica | 10.0 | 10.0 | 10.0 | 10.0 | 10.0 | 10.0 | 10.0 |
| | Silica | 2.0 | 2.0 | 2.0 | 2.0 | 2.0 | 2.0 | 2.0 |
| | Silicone elastomer | 3.0 | 3.0 | 3.0 | 3.0 | 3.0 | 3.0 | 3.0 |
| | Silicone elastomer- | 5.0 | 5.0 | 5.0 | 5.0 | 5.0 | 5.0 | 5.0 |
| | coated | | | | | | | |
| | Coated mica | 5.0 | 5.0 | 5.0 | 5.0 | 5.0 | 5.0 | 5.0 |
| | Preservative | 1.0 | 1.0 | 1.0 | 1.0 | 1.0 | 1.0 | 1.0 |
| | Magnesium stearate | 1.0 | 1.0 | 1.0 | 1.0 | 1.0 | 1.0 | 1.0 |
| | Colorant | 1.0 | 1.0 | 1.0 | 1.0 | 1.0 | 1.0 | 1.0 |
| | Mica & Titanium | 7.0 | 7.0 | 7.0 | 7.0 | 7.0 | 7.0 | 7.0 |
| | dioxide | | | | | | | |
| | Boron nitride | 1.0 | 1.0 | 1.0 | 1.0 | 1.0 | 1.0 | 1.0 |
| Raw | Ethylene/propylene/styrene | 7.5 | 7.5 | 7.5 | 7.5 | 7.5 | 7.5 | 7.5 |
| material 2 | copolymer | | | | | | | |
| | Butylene/ethylene/styrene | | | | | | | |
| | copolymer | | | | | | | |
| | Hexyl laurate | 5.0 | 5.0 | 5.0 | 5.0 | 5.0 | 5.0 | 5.0 |
| | Hydrogenated C6-14 | 9.0 | 9.0 | 9.0 | 9.0 | 9.0 | 9.0 | 9.0 |
| | olefin polymer | | | | | | | |
| | Diisostaryl maleate | 9.0 | 9.0 | 9.0 | 9.0 | 9.0 | 9.0 | 9.0 |
| | Silicone oil | 0.0 | 5.0 | 7.5 | 10.0 | 13.5 | 16.0 | 20.0 |

### Examples 8-10 and Comparative Example 8-11

### Preparation of Cosmetic Composition (Blusher)

The same materials as Examples 1-4 are used to provide a blusher, except that the content of a hydrocarbon gel (ethylene/propylene/styrene copolymer, butylene/ethylene/styrene copolymer) used in raw material 2 is changed.

The amounts of ingredients forming the blushers according to Examples and Comparative Examples are shown in the following Table 4 in more detail. The blushers are obtained by using the ingredients as described below in the same manner as described above.

TABLE 4
Blusher Compositions

| | | | | | | | Comp. | Comp. |
|---|---|---|---|---|---|---|---|---|
| | | Comp. | Comp. | | | Ex. | Ex. | Ex. |
| | | Ex. 8 | Ex. 9 | Ex. 8 | Ex. 9 | 10 | 10 | 11 |
| Raw | Talc | To 100 | To 100 | To 100 | To 100 | To 100 | To 100 | To 100 |
| material 1 | Mica | 10.0 | 10.0 | 10.0 | 10.0 | 10.0 | 10.0 | 10.0 |
| | Silica | 2.0 | 2.0 | 2.0 | 2.0 | 2.0 | 2.0 | 2.0 |
| | Silicone elastomer | 3.0 | 3.0 | 3.0 | 3.0 | 3.0 | 3.0 | 3.0 |
| | Silicone elastomer- | 5.0 | 5.0 | 5.0 | 5.0 | 5.0 | 5.0 | 5.0 |
| | coated | | | | | | | |
| | Coated mica | 5.0 | 5.0 | 5.0 | 5.0 | 5.0 | 5.0 | 5.0 |
| | Preservative | 1.0 | 1.0 | 1.0 | 1.0 | 1.0 | 1.0 | 1.0 |
| | Magnesium stearate | 1.0 | 1.0 | 1.0 | 1.0 | 1.0 | 1.0 | 1.0 |
| | Colorant | 1.0 | 1.0 | 1.0 | 1.0 | 1.0 | 1.0 | 1.0 |
| | Mica & Titanium | 7.0 | 7.0 | 7.0 | 7.0 | 7.0 | 7.0 | 7.0 |
| | dioxide | | | | | | | |
| | Boron nitride | 1.0 | 1.0 | 1.0 | 1.0 | 1.0 | 1.0 | 1.0 |
| Raw | Ethylene/propylene/ | 0.0 | 5.0 | 7.0 | 10.0 | 12.0 | 15.0 | 20.0 |
| material 2 | styrene copolymer | | | | | | | |
| | Butylene/ethylene/styrene | | | | | | | |
| | copolymer | | | | | | | |
| | Hexyl laurate | 5.0 | 5.0 | 5.0 | 5.0 | 5.0 | 5.0 | 5.0 |
| | Hydrogenated C6-14 | 9.0 | 9.0 | 9.0 | 9.0 | 9.0 | 9.0 | 9.0 |
| | olefin polymer | | | | | | | |
| | Diisostaryl maleate | 9.0 | 9.0 | 9.0 | 9.0 | 9.0 | 9.0 | 9.0 |
| | Silicone oil | 10.0 | 10.0 | 10.0 | 10.0 | 10.0 | 10.0 | 10.0 |

### Test Example 1

### Determination of Feel of Use and Molding Stability

Blushers are prepared according to the ingredients and mixing ratios as shown in Tables 2, 3 and 4 and are determined in terms of a feel of use and molding stability. To determine a feel of use, 25 panels are selected and provided with the following samples. Each panel is allowed to use the samples around her cheek and evaluate the samples, wherein 5 points are rated as perfect. The evaluation results are averaged.

To evaluate a feel of use, applicability, dust flying, adhesion, elastic feel, jamming and spreadability are determined. To determine molding stability, dropping stability is also determined. Five females of 20-30 ages participate in evaluating a feel of use and the test results are averaged. Applicability is determined through a feel (soft, rough, etc.) when each sample is applied to an eye rim or cheek. Dust flying is determined by measuring a degree of dropping of each sample while it flies in the form of dust, when applying the sample to an eye rim or cheek. Adhesion is determined by measuring a degree of staining of tissue with each sample after wiping the sample applied to an eye rim or cheek with tissue. An elastic feel is determined by measuring the softness of a blusher composition when pushing it with a forefinger lightly. Jamming is measured by measuring the amount of sample jammed in wrinkles of an eye rim or cheek one hour after applying the sample thereto. Spreadability is determined by measuring how each sample is spread naturally toward the application site when the sample is applied to an eye rim or cheek. In addition, dropping stability is measured by dropping a molded product sample from a height of 70 cm vertically to a 3 mm rubber plate three times. The test results are shown in the following Tables 5-7.

TABLE 5
Blusher Compositions of Examples 1-4 and Comparative Examples 1-3

| | Comp. | | | Comp. | | | Comp. |
|---|---|---|---|---|---|---|---|
| Item | Ex. 1 | Ex. 1 | Ex. 2 | Ex. 2 | Ex. 3 | Ex. 4 | Ex. 3 |
| Applicability | 3 | 4 | 3.5 | 2.5 | 3.5 | 3 | 2.5 |
| Dust flying | 5 | 4.5 | 3 | 2 | 4 | 3 | 2 |
| Adhesion | 5 | 4.5 | 3 | 2 | 4 | 3 | 2 |
| Elastic feel | 2 | 3.5 | 4 | 5 | 3.5 | 4.5 | 4.5 |
| Jamming | 5 | 4 | 3 | 2 | 4 | 3 | 2.5 |
| Spreadability | 2 | 3 | 4 | 4 | 4 | 4.5 | 4.5 |
| Dropping | 5 | 4 | 3 | 1 | 3.5 | 3 | 2 |
| stability | | | | | | | |

5: Excellent,
4: good,
3: satisfactory,
2: bad,
1: poor
Determination of Hardness: rheometer and A type hardness tester used (Adapter: #6(5Φ), S. ADJ: 15, S range: 0.1 mm, Range: 500, T. speed: 2 cm/min.)

After determination, it can be seen that the blusher composition of Comp. Ex, 1 has lower elastic feel, spreadability and applicability as compared to that of Examples. The blusher composition of Comp. Ex. 2 shows a low rating in terms of dust flying, adhesion, dropping stability and jamming. In addition, the composition of Comp. Ex. 3 has good spreadability and elastic feel, but shows a low rating in terms of jamming and adhesion with time. On the contrary, the blusher compositions of Examples 1-4 provide good results after evaluating elastic feel, dropping stability, jamming and adhesion. Particularly, the blusher compositions of Examples 3 and 4 using a coated silicone elastomer shows better elastic feel and spreadability as compared to the compositions of Examples 1 and 2.

TABLE 6
Blusher Compositions of Examples 5-7 and Comparative Examples 4-7

| | Comp. | Comp. | | | | Comp. | Comp. |
|---|---|---|---|---|---|---|---|
| Item | Ex. 4 | Ex. 5 | Ex. 5 | Ex. 6 | Ex. 7 | Ex. 6 | Ex. 7 |
| Applicability | 2.0 | 3.0 | 3.5 | 4.0 | 4.0 | 3.0 | 2.5 |
| Dust flying | 4.0 | 3.5 | 3.5 | 3.5 | 4.0 | 3.0 | 2.5 |
| Adhesion | 3.0 | 3.5 | 3.5 | 4.0 | 4.0 | 3.5 | 3.0 |
| Elastic feel | 1.0 | 2.5 | 3.5 | 4.0 | 4.5 | 4.5 | 4.5 |
| Jamming | 4.0 | 3.5 | 3.5 | 3.5 | 3.5 | 2.0 | 1.5 |
| Spreadability | 2.0 | 3.0 | 3.5 | 4.0 | 3.5 | 2.5 | 2.0 |
| Dropping | 4.0 | 3.5 | 3.5 | 4.0 | 3.5 | 3.0 | 2.0 |
| stability | | | | | | | |

5: Excellent,
4: good,
3: satisfactory,
2: bad,
1: poor
Determination of Hardness: rheometer and A type hardness tester used (Adapter: #6(5Φ), S. ADJ: 15, S range: 0.1 mm, Range: 500, T. speed: 2 cm/min.)

After determination, it can be seen that the blusher composition of Comp. Ex, 4 has lower elastic feel, spreadability and applicability as compared to the blusher compositions of Examples. The blusher composition of Comp. Ex. 5 shows a good result in terms of dust flying, but a low rating in terms of elastic feel and spreadability. In addition, the compositions of Comp. Ex. 6 and Comp. Ex. 7 have good elastic feel, but shows a low rating in terms of jamming, spreadability and dropping stability. On the contrary, the blusher compositions of Examples 5-7 provide good results in all of the evaluation items.

TABLE 7
Blusher Compositions of Examples 8-10 and Comparative Examples 8-11

| | Comp. | Comp. | | | Ex. | Comp. | Comp. |
|---|---|---|---|---|---|---|---|
| Item | Ex. 8 | Ex. 9 | Ex. 8 | Ex. 9 | 10 | Ex. 10 | Ex. 11 |
| Applicability | 4.0 | 3.5 | 3.5 | 3.5 | 3.0 | 2.5 | 2.0 |
| Dust flying | 2.0 | 2.5 | 3.0 | 3.0 | 3.5 | 3.5 | 4.0 |
| Adhesion | 1.0 | 2.0 | 3.0 | 3.5 | 3.5 | 3.5 | 3.5 |
| Elastic feel | 1.5 | 2.5 | 3.0 | 3.5 | 3.0 | 3.5 | 4.0 |
| Jamming | 4.5 | 4.0 | 4.0 | 3.5 | 3.0 | 2.5 | 1.0 |
| Spreadability | 4.0 | 3.5 | 3.5 | 3.0 | 3.0 | 2.5 | 2.0 |
| Dropping | 1.0 | 2.5 | 3.0 | 3.5 | 4.0 | 4.5 | 4.5 |
| stability | | | | | | | |

5: Excellent,
4: good,
3: satisfactory,
2: bad,
1: poor
Determination of Hardness: rheometer and A type hardness tester used (Adapter: #6(5Φ), S. ADJ: 15, S range: 0.1 mm, Range: 500, T. speed: 2 cm/min.)

After determination, it can be seen that the blusher compositions of Comp. Ex. 8 and Comp. Ex. 9 have lower adhesion, elastic feel and dropping stability as compared to the blusher compositions of Examples. The blusher compositions of Comp. Ex. 10 and Comp. Ex. 11 show a good result in terms of adhesion, elastic feel and dropping stability, but a low rating in terms of applicability, jamming and spreadability. On the contrary, the blusher compositions of Examples 8-10 provide good results in all of the evaluation items.

### INDUSTRIAL APPLICABILITY

As described above, according to the present invention, a silicone elastomer of a surface-treated powder type and a polymer are used to improve the dispersibility of silicone elastomer when producing a cosmetic composition. As a result, it is possible to provide a make-up cosmetic composition having a silky and soft elastic feel. The resultant cosmetic composition is adhered to the skin smoothly, has a good elastic feel and spreadability and shows little dust flying.

1. A make-up cosmetic composition, comprising 2-9 wt % of a powder type silicone elastomer, 40-50 wt % of a powder extender pigment, 7-12 wt % of a hydrocarbon gel, 7-14 wt % of anhydrous silicone oil, and 20-25 wt % of organic dispersant oil.

2. The make-up cosmetic composition according to claim 1, wherein the powder type silicone elastomer is a silicone elastomer surface-treated with silica, lauroyl lysine, alumina or titanium dioxide.

3. The make-up cosmetic composition according to claim 1, wherein the powder extender pigment includes at least one selected from the group consisting of talc, mica, silica, titanium dioxide, zinc oxide, boron nitride, pearlescent pigments and dyes.

4. The make-up cosmetic composition according to claim 1, wherein the hydrocarbon gel is one obtained by dispersing an ethylene/propylene/styrene copolymer or butylene/ethylene/styrene copolymer into a mineral oil, isoparaffin or isopropyl palmitate solvent.

5. The make-up cosmetic composition according to claim 1, wherein the hydrocarbon gel has a viscosity of 50,000-70,000 cPs.

6. The make-up cosmetic composition according to claim 1, wherein the anhydrous silicone oil is a dimethicone/vinyl dimethicone crosspolymer dispersed in dimethicone.

7. The make-up cosmetic composition according to claim 1, wherein the organic dispersant oil is ester-based oil and/or olefin oil.

8. The make-up cosmetic composition according to claim 1, which is obtained by mixing the ingredients together to form elastic flour slurry and molding the slurry into a formulation having a desired shape by using an extruder or molding machine.

9. The make-up cosmetic composition according to claim 1, which is a gel type or jelly type composition.

10. The make-up cosmetic composition according to claim 1, which is a blusher or eye shadow.
